(19) United States
(12) Patent Application Publication
(10) Pub. No.: US 2021/0375278 A1
(43) Pub. Date: Dec. 2, 2021

(54) SYSTEM AND METHOD FOR PROVIDING A HEALTH CARE RELATED SERVICE

(71) Applicant: Universal Electronics Inc., Santa Ana, CA (US)

(72) Inventors: Arsham Hatambeiki, Santa Ana, CA (US); Menno Koopmans, Santa Ana, CA (US); Lee Haughawout, Santa Ana, CA (US); Dai Tanaka, Santa Ana, CA (US)

(21) Appl. No.: 16/889,984

(22) Filed: Jun. 2, 2020

(51) Int. Cl. G10L 15/22; G10L 25/66; G08B 21/02; F24F 11/50; G16H 50/20; G16H 50/30; G16H 10/60; G05B 19/042

(52) U.S. Cl. CPC G10L 15/22; G10L 25/66; G08B 21/02; F24F 11/50; G05B 2219/2614; G16H 50/30; G16H 10/60; G05B 19/042; G16H 50/20

(57) ABSTRACT

A system for providing a health care service includes a sound detection device, a first environmental sensor, a network accessible platform, and a network communicatively coupling the sound detection device, the first environmental sensor, and the network accessible platform. The network accessible platform is programmed to evaluate a sound data received from the sound detection device and generated by a user and a first sensor data received from the first environmental sensor to determine an abnormal condition associated with the user. The platform then causes an action to be performed by at least one further device coupled to the network accessible platform. The action is an action that was associated with the determined abnormal condition associated with the user.

### BACKGROUND

Virtual health care services are generally known in the art. For example, US Publication No. 2018/0068082 (which publication is incorporated herein by reference in its entirety) describes a conversation user interface that enables patients to better understand their healthcare by integrating diagnosis, treatment, medication management, and payment, through a system that uses a virtual assistant to engage in conversation with the patient. The conversation user interface conveys a visual representation of a conversation between the virtual assistant and the patient. An identity of the patient, including preferences and medical records, is maintained throughout all interactions so that each aspect of this integrated system has access to the same information. The conversation user interface allows the patient to interact with the virtual assistant using natural language commands to receive information and complete tasks related to his or her healthcare.

As further described, a speech recognition engine associated with the conversation user interface may be equipped with additional algorithms for understanding the speech of the patient when his or her voice is modified by a health condition, such as a stuffy nose caused by a cold. Additionally, the speech recognition engine may have further algorithms to detect the effects of pain on speech patterns of the patient. If it is detected that the patient is in pain, this information may be used as context for interpreting queries and instructions from the patient. In some implementations, the speech recognition engine may reside at the virtual assistant, while in other implementations, functionality in the speech recognition engine is distributed at an electronic device and the virtual-assistant service. In other implementations, the speech recognition engine may be a third-party service that is leveraged by the virtual-assistant service and/or the electronic device.

### SUMMARY

The following describes improved systems and methods for providing a health care related service.

In one example, a system utilizes one or more devices having one or more microphones to receive user sound input. The device may be a far field voice detection device (such as “Amazon's Echo,” “Google's Google Home,” “Apple's Siri,” etc.), a sensor, a remote control, a home appliance, a stand-alone microphone, and/or the like (individually and collectively referred to hereinafter as a “sound detection device”). While not required, the sound detection device would typically be installed in the home, would be coupled to an existing home Wi-Fi network, and placed in a convenient location where the sound detection device may be used most frequently, such as in a family room, kitchen, baby's room, or the like.

In one example, one or more sound detection devices in a system accept audio input and the system determines, via voice learning, the end user's normal voice and tone. In this manner, when the user's voice changes, e.g., because of a sore throat, a change in mood (screaming or crying is detected), etc., the change in voice pattern is identified and data associated with this detected user event may be provided to a medical server, such as a third-party operated, on-line medical server, for evaluation. To this end, the medical server may have access to the user's medical history. The data associated with/collected because of the detected health event may be aggregated at the medical server for preventive healthcare and be made available to the user's medical practitioner.

In one example, a system is employed in an elderly care facility that already monitors the people under their care.

In one example, a voice enabled, remote-control of a system functions to perform any required voice activation which initiates a sound capturing process whereupon the captured sound data is processed by the system. The sound capturing process could be performed by the voice enabled, remote control device itself or an appliance, such as a set-top box, controllable via use of the remote-control device.

In one example, when a sound capture indicates an abnormal condition for a user, a system provides a notification (e.g., a text message, email, phone call, or the like) to a third-party (e.g., a parent, guardian, relation, care taker, etc.). The third party may then sign into/access a monitoring service or one of more devices in an environment to monitor a condition of the user.

In one example, a system operates in an environment comprising smart home sensors, such as those for detecting air quality (indoor and/or outdoor) which sensors provide input data to a device within the system for analysis. The data may be used to assist the system in diagnosing a condition for the user, e.g., to help differentiate between a cold, flu, and seasonal allergy.

In one example, a sound detecting device of a system determines to provide information to a monitoring health care service, directly to a third-party cell phone, e.g., via use of an app, or both. Similarly, the data may be determined to be provided to an intermediary device associated with a backend service server. Such a service may comprise pre-provisioning the in-home monitoring device using the authentication provided by the end user's health insurance carrier and the backend service links may be pre-established prior to the monitoring device shipping to the end user.

In one example, a sound detecting device in the system continually monitors in the background for key ambient audio signature, such as different types of cough, the tone of voice when speaking, etc.

In one example, a system tracks a normal (healthy) voice tone and compares that voice tone to changes in voice to identify signs of sickness.

In one example, a system has machine learning driven models to also identify different types of coughs, such as allergy, cold or flu, long time smoker, and even “nervous” coughs.

In one example, a system is used in the home to provide early notifications about a possibility of one or more health or allergy issues for one or more occupants of the home and to provide related health advice.

In one example, a system is used in an independent living facility for the elderly to provide early notifications to a resident health care provider, such as a nurse practitioner, to do a deeper review of a resident's health situation.

In one example, a system is used in a shared work facility, such as a factory or an office building, to detect possible health issues in general and/or for specific employees to trigger an alert for review for management, advice to leave common areas to prevent spread of possible disease, etc.

In one example, a system can take preemptive action to help make the home more comfortable for a resident, e.g., a toddler, elderly adult, etc., through a mixed use of data obtained by sensors in the environment, such as air quality sensors and humidity sensors, data retrieved from public air and health databases, e.g., those indicating outdoor AQI, or common diseases, data obtained from local air purifiers, humidifiers, heaters, A/C. units, etc. and/or data from other known sources of environmental information. In this manner, dependent on the parameters seen from local sensors and data obtained from cloud services, as well as the detected types of “cough” or “rough voice”, the system can adjust/control environmental conditions in the home, e.g., control temperature and/or humidity, enable purifiers, inject essential oils or disinfectants in the area, and/or the like, to both make it easier on the sick as well as prevent spread of disease.

In one example, a system uses an environmental sensor in the form of a pyroelectric (PIR) sensor, for example as found in a thermostat for hotels, to detect occupant temperature and report it back to a system server. In one example, this will allow the hotel to identify potentially sick occupants during or after checkout so they can target the room for disinfection, etc.

In one example, sensor data received by the PIR sensor is passed thru to an external server or cloud service for analysis where the data, which may comprise multiple samplings over a predetermined period of time, is analyzed to determine if the person has a fever.

In one example, a PIR sensor is installed in a single thermostat or in an intermediary device.

In one example, multiple PIR sensors are installed throughout the environment to capture multiple temperature samples.

In one example, a PIR sensor is in a camera or a set of cameras that send data to an intermediate device, such as a thermostat, for the intermediate device to use, as needed, and for the intermediate device to send to a target monitoring server or cloud service, as desired.

A better understanding of the objects, advantages, features, properties and relationships of the hereinafter disclosed systems and methods for providing a health care related service will be obtained from the following detailed description and accompanying drawings which set forth illustrative examples and which are indicative of the various ways in which the principles of the described systems and methods may be employed.

### BRIEF DESCRIPTION OF THE DRAWINGS

The features, advantages, and objects of the subject disclosure will become more apparent from the detailed description as set forth below, when taken in conjunction with the drawings in which like referenced characters identify correspondingly throughout, and wherein:

FIG. 1 illustrates example elements of a system for providing a health care related service;

FIG. 2 illustrates example elements of another system for providing a health care related service;

FIG. 3 illustrates example elements of a sound detection device of FIGS. 1 and 2;

FIG. 4 illustrates example elements of a command response system/voice processing system of FIGS. 1 and 2; and

FIG. 5 illustrates example steps performed by the systems illustrated in FIGS. 1 and 2.

### DETAILED DESCRIPTION

Devices having microphones are becoming commonplace in today's homes. Products such as far field voice detection devices (e.g., “Amazon's Echo,” “Google's Google Home,” “Apple's Siri,” etc.), cable service provider remote controls, and the like are all examples of these devices. Typically, such devices (collectively and individually referred to hereinafter as a “sound detecting device”) are installed at home, coupled to an existing home Wi-Fi network and placed in a convenient location where they may be used most frequently, such as in a family room, bedroom, or kitchen. An example of a sound detecting device in the form of a field voice detection device system is described in U.S. Pat. No. 9,947,333, the disclosure of which is incorporated herein by reference in its entirety.

Whether embodied as a stand-alone device or embedded into another device, a sound detecting device generally listens for a wake-word to be spoken, such as “Alexa” for “Amazon's Echo” and “OK Google” for “Google's Home.” The wake-word is typically followed by a question or a command. The question or command that follows the wake-word is captured by the sound detecting device and is usually sent over the Internet to a voice recognition service that interprets the question or command and provides a response that is sent back over the Internet to the assistant (and/or to another designated device) for verbal playback (via a speaker that is typically integrated into each device) and/or for causing some commandable action to occur (such as lighting lights, playing music, etc.).

In the present instance, a sound detecting device is utilized to capture sounds in an environment whereupon the sounds are processed to provide health care related services. The devices can be enabled to capture such sounds following the utterance of a wake word, by simply being turned on, or otherwise as desired. By way of example, and without limitation, a health care related service can include one or more of an initiation of a medical query and answer session with a virtual medical assistant (or live person as appropriate), determining a treatment regimen, ordering of medication, contacting and working with health insurance, contacting and working with a monitoring service, controlling devices/objects in an environment, and the like.

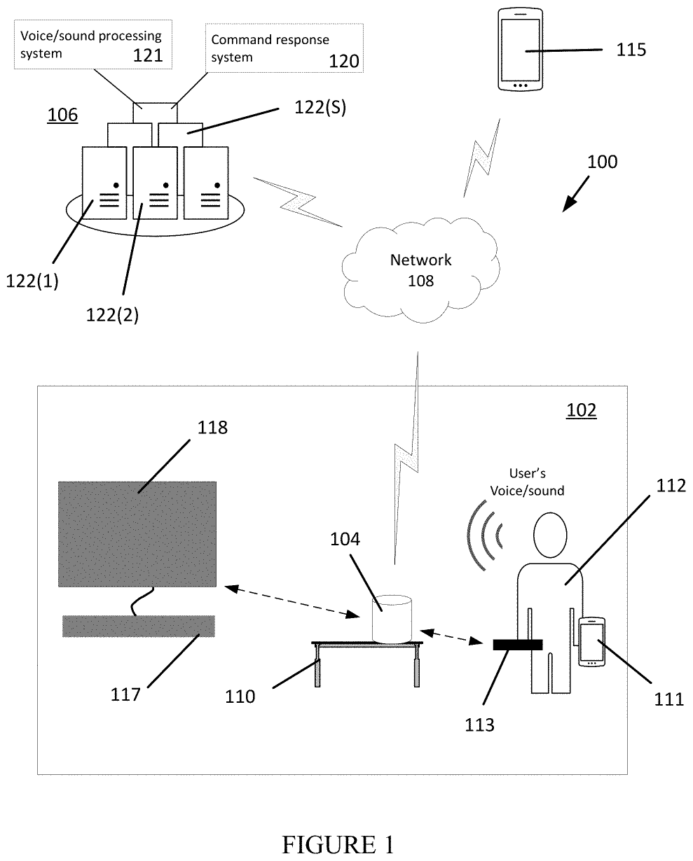

FIG. 1 shows an example sound detection system 100 including a device having a microphone, i.e., a sound detection device. The sound detection device may be a far field voice detection device 104, a remote control 113, a smart phone 111, an appliance 117/118, or the like without limitation. The sound detection device may also be a stand-alone microphone that, in turn, is coupled to another device.

The sound detection device is set in an exemplary home environment in which the sound detection device is physically situated in a room 102 of a home, hotel/motel, or the like. The sound detection device is communicatively coupled to one or more cloud-based services 106 over a network 108. The network 108 may include a local area network as well as a wide area network. Processing of sounds captured by the sound detection system 100 may be processed at the one or more cloud-based services 106, at the sound detecting device, or a combination of both. In the event the sound detection device is capable of fully processing captured sounds, it will be appreciated that the sound detecting device need not be connected to external servers/services.

In the illustrated example, the sound detection device 104 is depicted as a stand-alone device that is positioned on a table 110 within the room 102. In other examples, the sound detection device 104 may be placed in any number of locations. While the sound detection device 104 is illustrated as a stand-alone device, it will be appreciated that a sound detection device may be integrated into other devices within the home, that more than one sound detection device may be positioned in a single room or environment, one sound detection device may be used to accommodate user interactions from more than one room, that sound processing may be distributed amongst multiple sound detection devices, and the like.

Generally, the sound detection device has at least a plurality of microphones and a speaker to facilitate audio interactions with a user 112. The sound detection device may additionally include, as needed for a given purpose, a keyboard, a keypad, a touch screen, a joystick, control buttons, a display, and/or the like. In certain implementations, a limited set of one or more input elements may be provided. For example, the sound detection device may include a dedicated button to initiate a configuration process, to power on/off the device, to control output volume levels, etc. Nonetheless, the primary (and potentially only) mode of user interaction with the sound detection device is through voice input and audible, display, and/or command transmission output.

As noted, the plurality of microphones 214 of the sound detection device are provided to detect words and sounds from the user 112. Typically, the sound detection device, particularly when in the form of the far field voice detection device 104, uses the microphones 214 to listen for a predefined wake-word and, after the predefined wake-work is detected, the sound detection device uses the microphones 214 to listen for (and capture) sounds, questions, answers, and/or commands that are subsequently generated by/uttered from the user 112. Generally, the sounds, questions, answers, and/or commands that are received by the sound detection device via the one or more microphones are transmitted over the network 108 to the cloud services 106 for interpretation and subsequent action. In this regard, the sounds and utterances are particularly analyzed to perform health care related services.

The sound detection device may be communicatively coupled to the network 108 via use of wired technologies (e.g., wires, USB, fiber optic cable, etc.), via use of wireless technologies (e.g., RF, cellular, satellite, Bluetooth, etc.), and/or via use of other connection technologies. The network 108 is representative of any type of communication network, including a data and/or voice network, and may be implemented using a wired infrastructure (e.g., cable, CATS, fiber optic cable, etc.), a wireless infrastructure (e.g., RF, cellular, microwave, satellite, Bluetooth, etc.), and/or other connection technologies. The network 108 carries data, such as audio data, between the cloud services 106 and the far field voice detection device 104.

The network 108 may also be used to couple a service provided by the system to an external user. For example, the system may provide a notification service (or otherwise communicate with a third-party notification service) that functions to provide a notification to an end user via use of a connected device 115 when a sound (including speech) captured by the sound detection device indicates an abnormal condition for a user. Such notification may be made via a text message, email, phone call, or the like. Preferably, a user 112 or the like would register the third party, connected device 115 with the system/notification service. In addition to specifying a preferred communication method(s), such registration may additionally entail specifying to the system the condition(s) that are to trigger the sending of a notification. In some situations, the sending of a notification may further include providing access—whether directly or via a link (which may require further user authentication)—to the microphone and/or speaker of the sound detection device and/or to a camera that is associated with the system to thereby allow for the third party to monitor and/or interact with the user 112. As will be appreciated, the third party may be a parent, guardian, relation, care taker, a professional service, and the like without limitation.

As known in the art, the cloud services 106 generally refer to a network accessible platform implemented as a computing infrastructure of processors, storage, software, data access, and so forth that is maintained and accessible via a network such as the Internet. In the illustrated, example system 100, the cloud services 106 include a voice/sound processing system 121 that is intended to receive sound data from the sound detection device, process the received sound data (which may include using the received sound data to train one or more AI models), and perform an action, as necessary, based upon the outcome of the received sound data processing. At a minimum, it is desired that the voice/sound processing system 121 process the received sound data to provide a health care related service as described herein. It may also be desirable for the system to be adapted to process the sound data to provide control services. The control services may be a part of the health care related services.

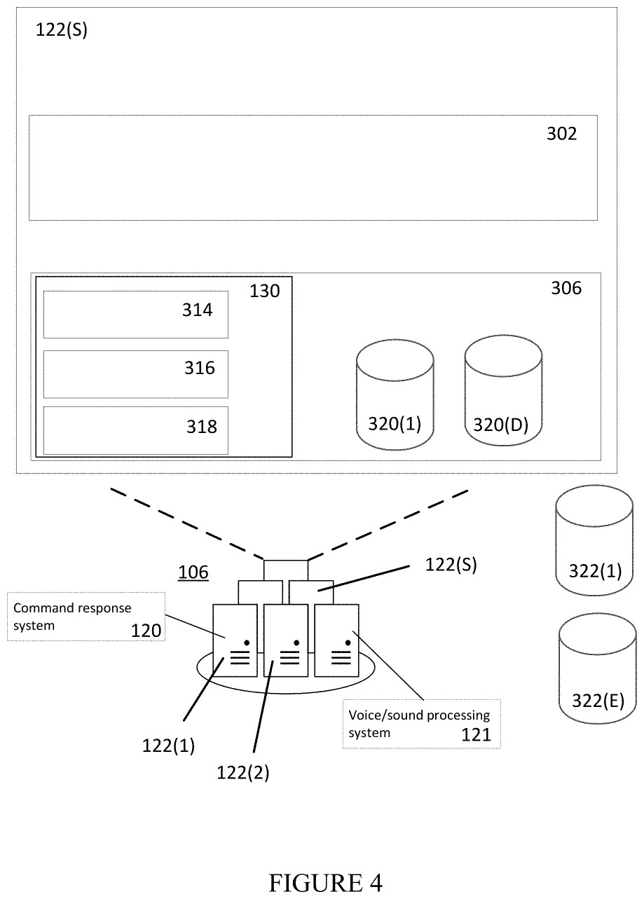

For providing control services, the cloud servers may further support a command response system 120. In any event, it will be appreciated that the servers 122(1)-(S) may host any number of applications/services that can process the user input received from the sound detection device and produce a suitable response. These servers 122(1)-(S) may be arranged in any number of ways, such as server farms, stacks, and the like that are commonly used in data centers. One example implementation of the voice/sound processing system 121 (which includes an optional command response system 120) is described below in more detail with reference to FIG. 4.

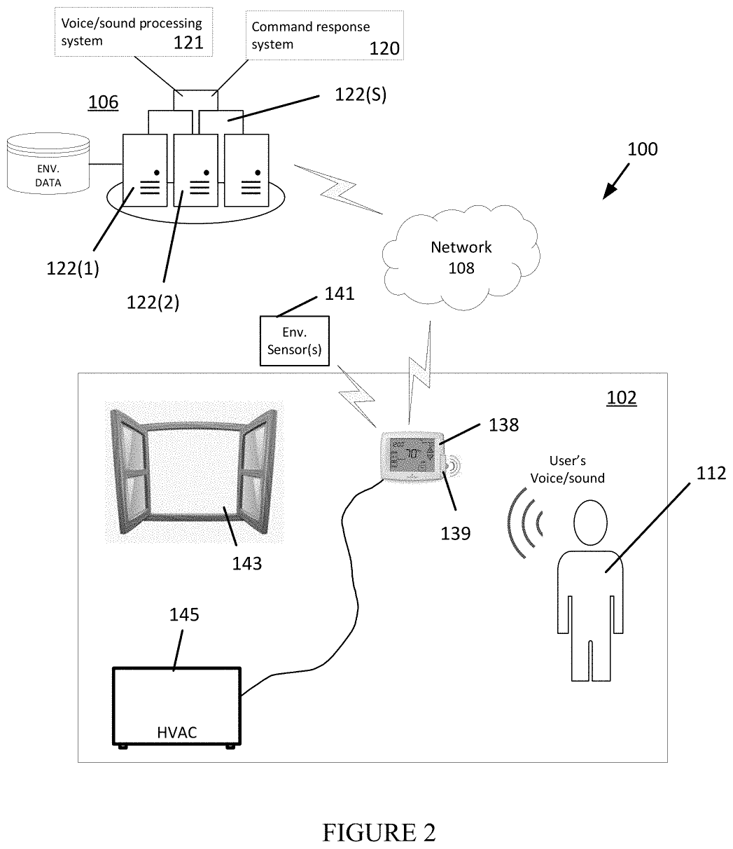

In some circumstances, such as illustrated in FIG. 2, the system 100 may also include a device having a sensor that is adapted to detect a temperature of the user 112. By way of example, the device may be a thermostat 138 that uses a pyroelectric (PIR) sensor 139, such as found in a thermostat for hotels to detect occupancy. The sensor may also be a component of a home security system or the like without limitation. In the system 100, the sensor 139 is used to detect the temperature of user 112 and report it back to a system server. The system server can then be programmed to perform an action based on the data collected by this environment sensor, i.e., the detected temperature of the user. For example, the server can send a notification to a third party to allow for monitoring of the user and/or to allow for other third-party action(s), e.g., to allow a hotel to identify a potentially sick occupant during or after checkout so they can target the room for disinfection. The server can cause further actions to be performed as well, such as causing a medication to be ordered for the user, causing a medical Q&A session to be commenced with the user, and the like as described herein.

It will be appreciated that the data captured by the sensor 139 may comprise multiple samplings over a predetermined period of time which multiple samplings would be analyzed to determine if the person has a fever. Likewise, while illustrated as a single PIR sensor 139 installed in a single device, the system 100 may employ multiple PIR sensors 139 installed throughout the environment to capture the multiple temperature samples. Yet further, the PIR sensor 139 can be a camera or a set of cameras that send data to an intermediate device, such as a thermostat, for the intermediate device to use, as needed, and for the intermediate device to send to a target monitoring server or cloud service, as desired.

In the system 100, data captured from the sound detection device, the user temperature sensor 139, additional, internal environment sensors (e.g., air temperature, humidity, and/or the like—which may be included as part of the thermostat 138), and/or external environmental sensors 141 (which external environmental data may also be obtained from a third-party system) can be processed by the system 100 to control various appliances, for example using the command response system 120. For example, the system 100 can take preemptive or responsive action to help make an environment more comfortable for a resident, e.g., a toddler, elderly adult, etc., through a mixed use of data obtained by the described devices/sensors in the environment and via use of AI associated with the medical service described herein. As noted such sensors may include, without limitation, air quality sensors and humidity sensors, data retrieved from public air and health databases, e.g., those indicating outdoor AQI, or common diseases, data obtained from local air purifiers, humidifiers, heaters, A/C. units, etc. and/or data from other known sources of environmental information. In addition, the system may include any captured sound information in the health service monitoring functions performed. In this manner, dependent on the parameters seen from local sensors and/or data obtained from cloud services, as well as the detected types of “cough” or “rough voice” from the user 112 and/or temperature of the user, the system 100 can cause commands to be issued to control the operation of one or more controllable devices to adjust/control environmental conditions in the home. For example, the system 100 can cause commands to be issue to the thermostat 138 (or other controlling device) to, in turn, control the state of one or more components of an HVAC system 145, e.g., to control air temperature and/or humidity, to enable purifiers, to inject essential oils or disinfectants in the area, to control a state of a window, e.g., to open or close windows 143, and/or the like, to both make it easier on the sick as well as prevent spread of disease.

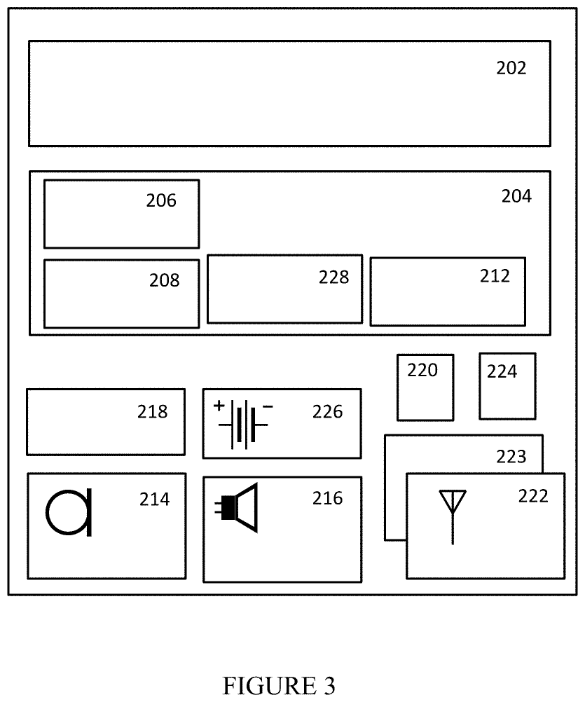

As shown in FIG. 3, selected functional components of an example sound detecting device are illustrated. In the illustrated example, the sound detection device includes a processor 202 and memory 204. The memory 204 may include computer-readable storage media (“CRSM”), which may be any available physical media accessible by the processor 202 to execute instructions stored on the memory. In one basic implementation, CRSM may include random access memory (“RAM”) and Flash memory. In other implementations, CRSM may include, but is not limited to, read-only memory (“ROM”), electrically erasable programmable read-only memory (“EEPROM”), or any other medium which can be used to store the desired information and which can be accessed by the processor 202.

Several modules such as instruction, datastores, and so forth may be stored within the memory 204 and configured to execute on the processor 202. An operating system module 206 is configured to manage hardware and services (e.g., a wireless unit, a USB unit, a Codec unit) within and coupled to the sound detection device. The sound detection device may also include a speech/sound recognition module 208 to provide some basic speech/sound recognition functionality. In some implementations, this functionality may be limited to specific commands that perform fundamental tasks like waking up the device, configuring the device, cancelling an input, and the like. In other instances, the sound recognition functionality may recognize a sound, such as cough, and may respond thereto by performing further actions, such as capturing more sounds, collecting more information from the user 112, etc. In any event, the amount of speech/sound recognition capabilities implemented on the sound detection device is an implementation detail, but the architecture described herein supports having some speech/sound recognition at the local, sound detection device together with more expansive speech recognition at the cloud services 106, for example as provided by the voice/sound processing system 121. A configuration module 212 may also be provided to assist in an automated initial configuration of the sound detecting device (e.g., to find a wifi connection, to enter login information, to link the far field voice detection device 104 to other devices, etc.) to enhance the user's out-of-box experience, as well as reconfigure the device at any time in the future.

In addition to the plurality of microphones 214 to receive audio input, such as user voice input, the sound detection device may have one or more speakers 216 to output audio sounds. A codec 218 may be coupled to the microphones 214 and the speaker 216 to encode and/or decode the audio signals as needed. The codec may convert audio data between analog and digital formats. A user may interact with the sound detection device by speaking to it, and the microphones 214 capture the user speech. The codec 218 encodes the user speech and transfers that audio data to other components. The sound detection device can communicate back to the user by emitting audible statements through the speaker 216. In this manner, the user may interact with the voice controlled assistant simply through speech and, in some instances, may allow a third party to interact with/monitor the user 112. To this end, the sound detection device may additionally include additional output and/or input devices, such as a display screen and/or a video or image capturing device.

In the illustrated example, the sound detection device includes a wireless unit 220 coupled to an antenna 222 to facilitate a wireless connection to a network, e.g., a home router, and an antenna 223 to facilitate a wireless connection to one or more other devices in the environment. The wireless unit 220 may implement one or more of various wireless technologies, such as wifi, Bluetooth (BLE), RF, and so on. The sound detection device and/or other devices in communication with the sound detection device may support Bluetooth (e.g., Bluetooth v 5.1) and may use an antenna 223 that will allow the sound detection device and/or the other devices to support direction finding functionality such as angle of arrival (“AoA”) direction finding functionality and/or angle of departure (“AoD”) direction finding functionality, as desired. It will be appreciated that devices that are intended to communicate with the sound detection device 104 may equally be provisioned with any hardware and software needed to support such direction-finding functionality. In some instances, such direction-finding functionality may be used to estimate the location of a user within an environment to thereby, for example, cause a camera to be oriented towards and/or to be focused on the user, to provide access to a select one of a plurality of cameras, and the like for monitoring purposes. As will also be appreciated, the microphones 214 could also be used to assist in sound based, direction-finding.

As additionally illustrated in FIG. 3, a USB port 224 may further be provided as part of the sound detecting device to facilitate a wired connection to a network or a plug-in network device that communicates with other wireless networks. In addition to the USB port 224, or as an alternative thereto, other forms of wired connections may be employed, such as a broadband connection, an HDMI connection, etc. A power unit 226 is further provided to distribute power to the various components on the sound detection device.

The sound detection device may also include a command transmission unit 228 which command transmission unit 228 will operate, in connection with antennas 222, 223, USB port 224, and/or other transmissions devices (such as an IR transmitter, a power line transmitter, etc.), to cause appropriate commands to be issued to one or more target appliances/devices to thereby control functional operations of such target appliances, e.g., to open/close a window, to turn on/off a HVAC system component, etc. A device having such control capabilities is described in U.S. application Ser. No. 104 16/717,546, the disclosure of which is incorporated herein by reference in its entirety. As additionally needed for any purpose, the sound detection device may include one or more sensors, such as the environmental sensors. It will also be appreciated that the thermostat 138 of FIG. 2 would likewise include one or more components shown in FIG. 3 as needed to perform the functions that are required thereof.

FIG. 4 shows selected functional components of a server architecture implemented by the voice/sound processing system 121 and, if included, the command response system 120 as part of the cloud services 106 of FIGS. 1 and 2. The systems 120/121 includes one or more servers, as represented by servers 122(1)-(S). The servers collectively comprise processing resources, as represented by processors 302, and memory 306. The memory 306 may include volatile and nonvolatile memory, removable and non-removable media implemented in any method or technology for storage of information, such as computer-readable instructions, data structures, program modules, or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information and which can be accessed by a computing device.

In the illustrated implementation, a processing module 130 is shown as software components or computer-executable instructions stored in the memory 306 and executed by one or more processors 302. The processing module 130 generally includes a speech/sound recognition engine 314, an optional command handler 316, and a response encoder 318. The speech/sound recognition engine 314 evaluates a sound received from a sound detector device to thereby provide health related services. In some instances, the speech/sound recognition engine may convert a user command to a text string. In this text form, the user command can be used in search queries, or to reference associated responses, or to direct an operation, or to be processed further using natural language processing techniques, or so forth. In other implementations, the user command may be maintained in audio form, or be interpreted into other data forms.

In the case where the system supports commandable actions, the user command is passed to a command handler 316 in its raw or a converted form, and the handler 316 performs essentially any operation that might use the user command as an input. As one example, a text form of the user command may be used as a search query to search one or more databases, such as internal information databases 320(1)-320(D) or external third part data providers 322(1)-322(E). Alternatively, an audio command may be compared to a command database (e.g., one or more information databases 320(1)-(D)) to determine whether it matches a pre-defined command. If so, the associated action or response may be retrieved. In yet another example, the handler 316 may use a converted text version of the user command as an input to a third-party provider (e.g., providers 322(1)-(E)) for conducting an operation, such as a financial transaction, an online commerce transaction, and the like.

In some instances, the system may generate a command for the command handler 316 based upon data received from one or more external devices/sensors. Accordingly, the system need not be limited to acting only upon receiving a voice command from a user 112. For example, the system might generate a command for the purpose of ultimately controlling a home appliance/device and/or to cause a notification to be sent to a third party based up the detection of a cough, the detection of agitation in the user's voice, abnormal environmental conditions within the household, and the like. For example, the system may cause a window to be closed, heat to be turned on, and the like based upon the information collected and as determined by the AI of the system.

It will also be appreciated that any operation may produce a response. When a response is produced, the response encoder 318 encodes the response for transmission back over the network 108 to the sound detection device and/or to another device that the system is aware of. In some implementations, this may involve converting the response to audio data that can be played at the sound detection device for audible output through the speaker to the user (e.g., to convey diagnosis and/or treatment information, to ask a question, etc.) or to command data that can be transmitted to a target appliance via use of a transmission protocol recognizable by the target appliance.

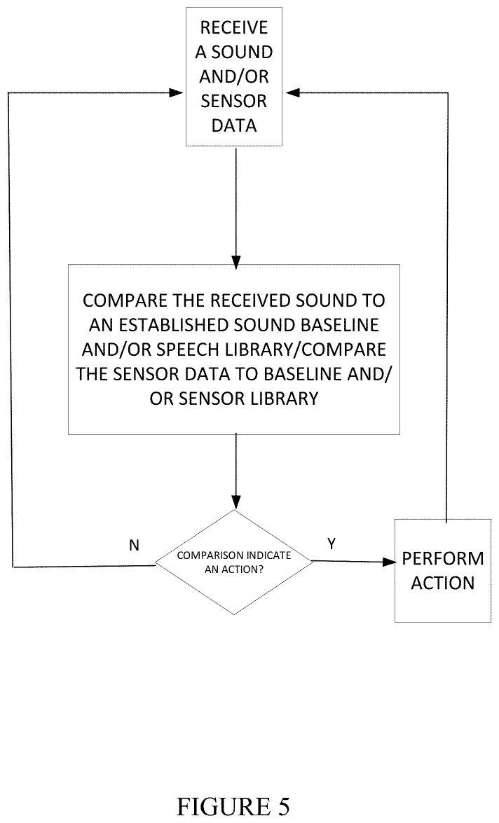

As shown in FIG. 5, the system generally functions to receive a sound (e.g., a cough or word(s) originating from a user) and/or data from a sensor (or other data source) whereupon the system will analyze the received sound by performing a comparison to one or more baselines established for the user and/or against one or more speech libraries and will compare the data to a baseline or threshold established for the corresponding sensor and/or data within a sensor data library. In this regard, it will be appreciated that such libraries and the like will function to correlate actions with determinative outcomes of one or more comparisons. Thus, when the analysis indicates that an action is to be performed, e.g., the system is to communicate further with the user, cause a command to be issued, transmit data to a healthcare related service/server, etc., such action is undertaken. In some instances, such action may include readying the system to receive and/or request more sound input from the user and/or data from one or more sensors.

While FIG. 5 shows a generalization of the processes that would be performed by the subject systems, it is to be understood that these processes, illustrated as a collection of blocks in a logical flow graph, represent operations that can be implemented in hardware, software, or a combination thereof. In the context of software, the blocks represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order or in parallel to implement the processes. It is understood that the following processes may be implemented with other architectures as well. In one example, one or more devices in a system accept audio input and the system determines, via voice learning, the end user's normal voice and tone. In this manner, when the user's voice changes, e.g., because of a sore throat, a change in mood (screaming or crying is detected), etc., the change in voice pattern is identified (via a comparison to the established normal(s) for the user), and the comparison may cause the system to perform one or more related actions. The action may be, for example, providing the sound data to a medical server, such as a third-party operated, on-line medical server, for evaluation. The medical server may have access to the user's medical history to thereby allow the server to further interact with the user (e.g., commence a question and answer session to collect yet further information for rendering a diagnosis, creating a treatment regimen, etc.). The data associated with/collected because of the detected health event may be aggregated at the medical server for preventive healthcare and be made available to the user's medical practitioner.

In one example, a system is employed in an elderly care facility that already monitors the people under their care and an action—caused to be initiated in response to a sound or data being determined to be abnormal—can be the sending of a notification to a health care providing employee of the facility. Likewise, when a sound capture indicates an abnormal condition for a user, a responsive action can cause the system to initiate the providing of a notification (e.g., a text message, email, phone call, or the like) to a third-party (e.g., a parent, guardian, relation, care taker, etc.). The notification may include a means for the third party to use one or more devices within the system o monitor a condition of the user.

In one example, a system operates in an environment comprising smart home sensors, such as those for detecting air quality (indoor and/or outdoor) which sensors provide input data to a device within the system for analysis. The data may be used to assist the system in diagnosing a condition for the user, e.g., to help differentiate between a cold, flu, and seasonal allergy, and/or may also be used to cause an environmental control action to be performed. The system can also take preemptive action to help make the home more comfortable for a resident, e.g., a toddler, elderly adult, etc., through a mixed use of data obtained by sensors in the environment, such as air quality sensors and humidity sensors, data retrieved from public air and health databases, e.g., those indicating outdoor AQI, or common diseases, data obtained from local air purifiers, humidifiers, heaters, A/C. units, etc. and/or data from other known sources of environmental information. In this manner, dependent on the parameters seen from local sensors and data obtained from cloud services, as well as the detected types of “cough” or “rough voice”, the system can adjust/control environmental conditions in the home, e.g., control temperature and/or humidity, enable purifiers, inject essential oils or disinfectants in the area, and/or the like, to both make it easier on the sick as well as prevent spread of disease.

In one example, a system has machine learning driven models to also identify different types of coughs, such as allergy, cold or flu, long time smoker, and even “nervous” coughs.

In one example, a system is used in the home to provide early notifications about a possibility of one or more health or allergy issues for one or more occupants of the home and to provide related health advice.

In one example, a system is used in a shared work facility, such as a factory or an office building, to detect possible health issues in general and/or for specific employees to trigger an alert for review for management, advice to leave common areas to prevent spread of possible disease, etc.

In view of the foregoing, it will be understood that the system may provide a virtual healthcare service that performs one or more of learning one or more characteristics about a user, learning one or more characteristics about an environment, receiving sound data captured from a user and environment data captured by one or more sensors, performing processing on any captured data, and determining one or more actions to perform based upon the processing. Such actions may the gathering of further information—medical and/or environmental, creating or employing a treatment regimen, contacting a health insurer and/or health care provider, performing a medical diagnosis; prescribing medication for the user, controlling one or more devices in a network, and the like without limitation.

While various concepts have been described in detail, it will be appreciated by those skilled in the art that various modifications and alternatives to those concepts could be developed in light of the overall teachings of the disclosure. For example, alternative thermal detectors, such as a thermopile device, may be used in place of or in combination with a PIR in any of the examples above. Further, while described in the context of functional modules and illustrated using block diagram format, it is to be understood that, unless otherwise stated to the contrary, one or more of the described functions and/or features may be integrated in a single physical device and/or a software module, or one or more functions and/or features may be implemented in separate physical devices or software modules. It will also be appreciated that a detailed discussion of the actual implementation of each module is not necessary for an enabling understanding of the invention. Rather, the actual implementation of such modules would be well within the routine skill of an engineer, given the disclosure herein of the attributes, functionality, and inter-relationship of the various functional modules in the system. Therefore, a person skilled in the art, applying ordinary skill, will be able to practice the invention set forth in the claims without undue experimentation. It will be additionally appreciated that the particular concepts disclosed are meant to be illustrative only and not limiting as to the scope of the invention which is to be given the full breadth of the appended claims and any equivalents thereof.

What is claimed is:

1. A system for providing a health care service, comprising:
a sound detection device;
a first environmental sensor;
a network accessible platform; and
a network communicatively coupling the sound detection device, the first environmental sensor, and the network accessible platform;
wherein the network accessible platform is programmed to evaluate a sound data received, via the network, from the sound detection device and generated by a user and a first sensor data received, via the network, from the first environmental sensor to determine an abnormal condition associated with the user and to cause an action to be performed by at least one further device coupled to the network accessible platform, the action having been associated with the determined abnormal condition associated with the user.

2. The system as recited in claim 1, wherein the first environmental sensor comprises a thermal detector and the first sensor data comprises data indicative of a sensed temperature of the user.

3. The system as recited in claim 2, wherein the action comprises causing the at least one further device to present an alarm indicative of the user being determined to be ill.

4. The system as recited in claim 2, wherein the thermal detector is a component part of a room thermostat.

5. The system as recited in claim 4, wherein the at least one further device comprises a server associated with a hospitality service provider.

6. The system as recited in claim 1, wherein the sound data comprises sound data indicative of a user cough.

7. The system as recited in claim 1, wherein the sound data comprises sound data indicative of a user speaking with an abnormal speech pattern.

8. The system as recited in claim 1, wherein sound data comprises sound data indicative of the user having cold or flu symptoms.

9. The system as recited in claim 1, wherein the first environmental sensor comprises an air temperature sensor.

10. The system as recited in claim 9, wherein the action comprises causing the at least one further device to control a state of a component of an HVAC system.

11. The system as recited in claim 9, wherein the action comprises causing the at least one further component to control a state of a window.

12. The system as recited in claim 1, wherein the first environmental sensor comprises an air quality sensor.

13. The system as recited in claim 12, wherein the action comprises causing the at least one further device to control a state of a component of an HVAC system.

14. The system as recited in claim 12, wherein the action comprises causing the at least one further device to control a state of a window.

15. The system as recited in claim 1, further comprising a second environmental sensor and wherein the network communicatively couples the sound detection device, the first environmental sensor, the network accessible platform, and the second environmental sensor and the network accessible platform is programmed to evaluate the sound data received, via the network, from the sound detection device and generated by the user, the first sensor data received, via the network, from the first environmental sensor, and a second sensor data, received via the network, from the second environmental sensor to determine the abnormal condition associated with the user.

16. The system as recited in claim 15, wherein the first environmental sensor is located within the environment and the second environmental sensor is located outside of the environment.

17. The system as recited in claim 16, wherein the action comprises causing the at least one further device to control a state of a component of an HVAC system.

18. The system as recited in claim 16, wherein the action comprises causing the at least one further device to control a state of a window.

19. The system as recited in claim 1, wherein the action to be performed by the at least one further device coupled to the network accessible platform comprises causing a notification to be sent to a third-party device registered with the system.

20. The system as recited in claim 19, the action to be performed by the at least one further device coupled to the network accessible platform comprises allowing the third-party device to have access to one or both of an input device and an output device associated with the sound detection device.
